(19) United States
(12) Patent Application Publication
(10) Pub. No.: US 2007/0299442 A1
(43) Pub. Date: Dec. 27, 2007

(54) Vertebral stabilizer

(75) Inventors: Lukas Eisermann, San Diego, CA (US); Larry McBride, Memphis, TN (US)

Correspondence Address: HAYNES AND BOONE, LLP, 901 Main Street, Suite 3100, Dallas, TX, 75202, US

(73) Assignee: SDGI Holdings, Inc., Wilmington, DE (US)

(21) Appl. No.: 11/474,662

(22) Filed: Jun. 26, 2006

(51) Int. Cl. A61F 2/30

(52) U.S. Cl. 606/61; 623/17.11

(57) ABSTRACT

A system for flexibly stabilizing a vertebral column in tensile and compressive loading by connecting a first and a second vertebrae includes first means for connecting to the first vertebra and second means for connecting to the second vertebra. A flexible connector is configured to extend from and connect the first means to the second means. The flexible connector may include first and second apertures for respectively attaching to the first and second means for connecting. Each aperture may include a reinforcement member therein.

### BACKGROUND

Severe back pain and nerve damage may be caused by injured, degraded, or diseased spinal joints and particularly, spinal discs. Current methods of treating these damaged spinal discs may include vertebral fusion, nucleus replacements, or motion preservation disc prostheses. Disc deterioration and other spinal deterioration may cause spinal stenosis, a narrowing of the spinal canal and/or the intervertebral foramen, that causes pinching of the spinal cord and associated nerves. Current methods of treating spinal stenosis include laminectomy or facet resection. Alternative and potentially less invasive options are needed to provide spinal pain relief.

### SUMMARY

In one aspect, this disclosure is directed to a system for flexibly stabilizing a vertebral column in tensile and compressive loading by connecting a first and a second vertebrae. The system includes first means for connecting to the first vertebra and second means for connecting to the second vertebra. A flexible connector is configured to extend from and connect the first means to the second means.

In a further aspect, the flexible connector may include first and second apertures for respectively attaching to the first and second means for connecting. Each aperture may include a reinforcement member therein.

In yet another aspect, the flexible connector may include flexibility affecting holes formed therein.

In yet another aspect, each of the first and second vertebra has a transverse process and a superior articular process. The first means may connect to the first vertebra in a position between the transverse process and the superior articular process. The second means may connect to the second vertebra in a position between the transverse process and the superior articular process.

In another aspect, a method of flexibly stabilizing vertebrae on a spinal column is disclosed. The method includes accessing vertebrae and installing vertebral fasteners on the pair of pedicles. A flexible connector may be placed to extend around an exterior of the vertebral fasteners to connect the pair of pedicles. The flexible connector may be secured to the vertebral fasteners. In one aspect, installing the vertebral fasteners may be accomplished with the transverse processes and the superior articular processes of the vertebrae intact.

In yet another aspect, this disclosure is directed to a system for flexibly stabilizing a vertebral column in tensile and compressive loading by connecting a first and a second vertebrae. The system includes first means for connecting to the first vertebra and second means for connecting to the second vertebra. A single flexible connector may be configured to extend from and connect the first means to the second means. The flexible connector may include a first end and a second end that is rounded to reduce occurrence of distress to tissue about the first and second means.

### BRIEF DESCRIPTION OF THE DRAWINGS

FIG. 1 is a pictorial representation of a vertebral column with a vertebral stabilizing system according to one embodiment of the present disclosure.

FIG. 2 is a pictorial representation of a close-up view of the vertebral stabilizing system of FIG. 1.

FIG. 3 is a pictorial representation of an exploded view of the vertebral stabilizing system of FIG. 1.

FIGS. 4A-4C are illustrations of a flexible connector of the vertebral stabilizing system of FIG. 1.

FIGS. 5-7 are illustrations of perspective views of exemplary flexible connectors according to other embodiments of the present disclosure.

### DETAILED DESCRIPTION

The present disclosure relates generally to the field of orthopedic surgery, and more particularly to systems and methods for stabilizing a spinal joint. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

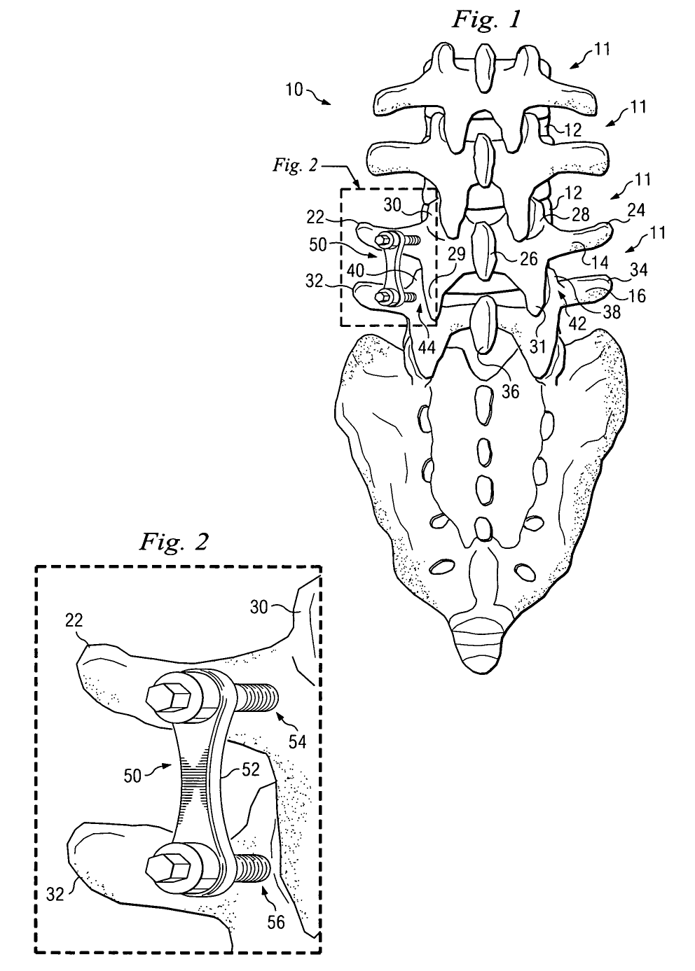

Referring to FIG. 1, the numeral 10 refers to a spinal column having a series of vertebral joints 11, each including an intervertebral disc 12. One of the vertebral joints 11 will be described further with reference to adjacent vertebrae 14, 16. The vertebra 14 includes transverse processes 22, 24; a spinous process 26; superior articular processes 28, 30; and inferior articular processes 29, 31. Similarly, the vertebra 16 includes transverse processes 32, 34; a spinous process 36; superior articular processes 38, 40; and inferior articular processes (not labeled). Although the illustration of FIG. 1 generally depicts the vertebral joint 11 as a lumbar vertebral joint, it is understood that the devices, systems, and methods of this disclosure may also be applied to all regions of the vertebral column, including the cervical and thoracic regions. Furthermore, the devices, systems, and methods of this disclosure may be used in non-spinal orthopedic applications.

A facet joint 42 is formed, in part, by the adjacent articular processes 29, 38. Likewise, another facet joint 44 is formed, in part, by the adjacent articular processes 31, 40. Facet joints also may be referred to as zygapophyseal joints. A healthy facet joint includes a facet capsule extending between the adjacent articular processes. The facet capsule comprises cartilage and synovial fluid to permit the articulating surfaces of the articular processes to remain lubricated and glide over one another. The type of motion permitted by the facet joints is dependent on the region of the vertebral column. For example, in a healthy lumbar region, the facet joints limit rotational motion but permit greater freedom for flexion, extension, and lateral bending motions. By contrast, in a healthy cervical region of the vertebral column, the facet joints permit rotational motion as well as flexion, extension, and lateral bending motions. As the facet joint deteriorates, the facet capsule may become compressed and worn, losing its ability to provide a smooth, lubricated interface between the articular surfaces of the articular processes. This may cause pain and limit motion at the affected joint. Facet joint deterioration may also cause inflammation and enlargement of the facet joint which may, in turn, contribute to spinal stenosis. Removal of an afflicted articular process may result in abnormal motions and loading on the remaining components of the joint. The embodiments described below may be used to stabilize a deteriorated facet joint while still allowing some level of natural motion.

Injury, disease, and deterioration of the intervertebral disc 12 may also cause pain and limit motion. In a healthy intervertebral joint, the intervertebral disc permits rotation, lateral bending, flexion, and extension motions. As the intervertebral joint deteriorates, the intervertebral disc may become compressed, displaced, or herniated, resulting in excess pressure in other areas of the spine, particularly the posterior bony elements of the afflicted vertebrae. This deterioration may lead to spinal stenosis. The embodiments described below may restore more natural spacing to the posterior bony elements of the vertebrae, decompress an intervertebral disc, and/or may relieve spinal stenosis. Referring still to FIG. 1, in one embodiment, a vertebral stabilizing system 50 may be used to provide support to the vertebrae 14, 16, decompress the disc 12 and the facet joint 44, and/or relieve stenosis.

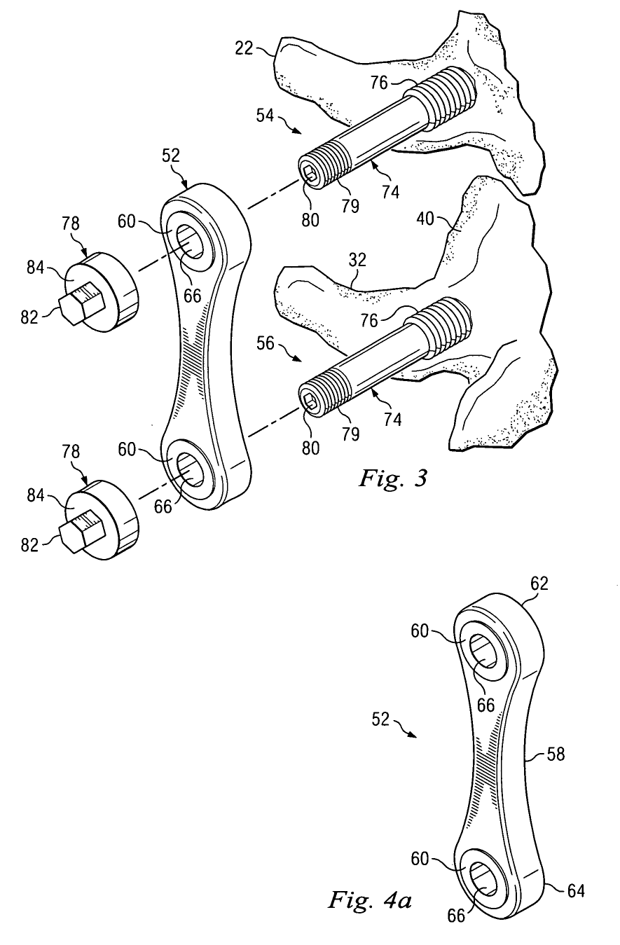

FIGS. 2 and 3 show the vertebral stabilizing system 50 disclosed in FIG. 1 in greater detail. FIG. 2 shows the system 50 assembled and FIG. 3 shows the system 50 in an exploded state. As shown in FIGS. 2 and 3, the vertebral stabilizing system 50 includes a flexible connector 52, a first vertebral fastener 54, and a second vertebral fastener 56.

Connected at each end to the vertebral fasteners 54, 56, the flexible connector 52 may provide compressive support and load distribution, providing relief to the intervertebral disc 12. In addition, the flexible connector 52 may dampen the forces on the intervertebral disc 12 and facet joint 44 during motion such as flexion. Because the flexible connector 52 is securely connected to the vertebral fasteners 54, 56, the flexible connector 52 also provides relief in tension. Accordingly, during bending or in extension, the flexible connector 52 may assist in providing a flexible dampening force to limit the chance of overcompression or overextension when muscles are weak. On top of this, the flexible connector 52 also allows torsional movement of the vertebra 14 relative to the vertebra 16.

The flexible connector 52 may be formed of an elastic, multi-directionally flexible material such as silicone, polyurethane, or hydrogel, which, in some embodiments, may be un-reinforced. In alternative embodiments, a flexible connector similar to connector 52 is reinforced to provide a desired stiffness. For example, in one exemplary embodiment, reinforcement fibers are uniformly disposed within the flexible connector. The fibers could be glass, carbon, or other material, preferably being biologically compatible. Further, desired fiber alignment may provide desired strengthening. For example, in one embodiment, fibers are aligned to strengthen and limit movement in the tension and/or compressive directions, while allowing near-un-reinforced levels of torsional movement. Other desired arrangements also may be provided by selectively aligning the reinforcing fibers. In some examples, the reinforcement is not uniform throughout the flexible connector. In one example, different regions of the flexible connector are reinforced while other regions are not, or alternatively, different regions of the flexible are reinforced by different amounts. In some examples, the regions of the flexible connector including aperatures 66 (described below) may be reinforced, while the central region of the flexible connector is not. In another exemplary embodiment, a flexible connector is reinforced through a vulcanization process. As would be apparent to one of ordinary skill in the art, others reinforcement methods also may be used, including a number of fiber lay-ups that maybe utilized to achieve various effects.

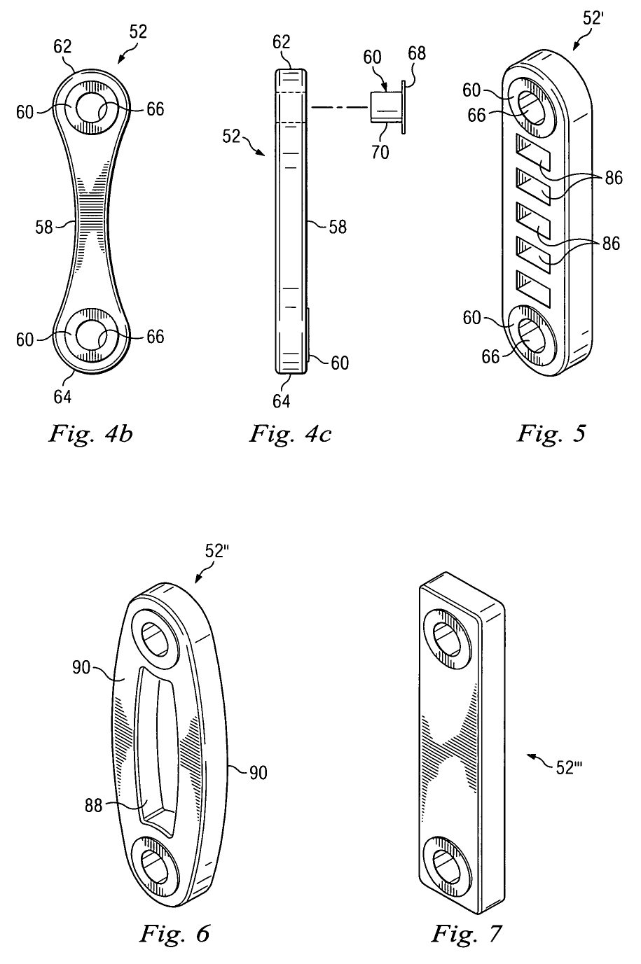

FIGS. 4A-4C show one exemplary embodiment of the flexible connector 52 in greater detail. In this embodiment, the flexible connector 52 includes a body 58 and two reinforcement members 60. The body 58 may be formed of a flexible material, extending between first and second ends 62, 64. The profile of the first and second ends 62, 64 may be rounded to reduce occurrence of distress to tissue about the ends, and to reduce occurrence of distress to tissue about the vertebral fasteners 54, 56. An aperture 66, adjacent each end 62, 64, is configured to interact with and connect to the vertebral fasteners 54, 56. The flexible connector 52 may be configured to have any desired tensile, torsional, and compressive properties, and in this embodiment, is designed in an hour-glass shape having a width thinner in the central regions than at the ends 62, 64. This design may provide desired torsional stiffness, while also providing a desired tensile stiffness.

The reinforcement members 60 are optional components that may be used to strengthen the apertures 66. In the embodiment shown, the reinforcement members 60 are grommets that fit within the apertures 66 and distribute loads from the vertebral fasteners 54, 56 to the flexible connector 52. In the exemplary embodiment shown, the grommets have a flange 68 at one side and a body 70 having a length substantially similar to the thickness of the flexible connector 52. Accordingly, when the grommet is placed within the aperture 66, the flange 68 may lie flat against the flexible connector 52, while the body 70 may extend substantially entirely through the aperture 66. Accordingly, the entire aperture 66 is reinforced with the reinforcing member 60.

It should be noted that the reinforcing member 60 may differ from that shown, so long as it provides an element of support or load distribution to the flexible member 52. For example, when the reinforcement member 60 is the disclosed grommet, a flange 68 may be disposed on each side of the flexible connector 52. In another embodiment, the grommet may extend only partially through the flexible connector 52. In one exemplary embodiment, the flexible connector 52 is formed to include a recess about the apertures 66 to receive the flange so that, when inserted into the aperture 66, the flange 68 of the reinforcement member 60 lies recessed into, flush with, or below the surface of the flexible connector 52. In other exemplary embodiments, the reinforcement member may be a tubular liner or, alternatively, a rivet. In other alternative embodiments, the reinforcement member is reinforcing thread, rope, or wire that may be sewn into the flexible connector about the apertures. The reinforcement members may be any member configured to reduce point loads or strengthen the apertures of the flexible connector.

As shown in FIG. 3, the vertebral fasteners 54, 56 are configured to attach to the vertebrae 14, 16 and provide an attachment location for the flexible connector 52. In the embodiment shown, the vertebral fasteners 54, 56 each include a screw 74 and a set screw 78.

In one embodiment, each screw 74 may include external threads 76 configured to embed in and secure the screw 74 to the bone. In some embodiments, the screws 74 may include perimeter threads 79 usable to attach to additional components of the vertebral fasteners 54, 56. For example, the perimeter threads 79 may be configured to engage the threads formed on the set screw 78. It should be noted that the screws 74 may be compatible with attachment devices that do not use a set screw, but use other means and systems for attaching the flexible connector 52 in place. In the exemplary embodiment shown in FIG. 3, the screw 74 includes a recessed hex head 80 for insertion. A hex tool (not shown) may be inserted into the recessed hex head 80 and turned to drive the screw 74 into place. The screw 74 may include additional features as would be apparent to one skilled in the art.

In FIG. 3, the screws 74 are driven into the pedicle at a location between both the transverse process 22 and the superior articular process 30 (shown in FIG. 2). More particularly, in the example shown, the screws 74 are driven adjacent the base of the transverse process 22 in the area between the transverse process 22 and the superior articular process 30. By inserting the screws 74 in this location, rather than removing a transverse or spinous process and placing the screw 74 in its location, the integrity of the vertebra 14 is maintained, reducing the chance of abnormal loading and motion of the remaining joint component. In other embodiments however, the screws 74 may be driven into the transverse or spinous processes themselves.

The set screw 78 may be configured to operate to secure the flexible connector 52 on the screw 74. In one embodiment, the set screw 78 may be configured to engage the perimeter of the screw 74. In this example, the set screw 78 includes an axially extending hex head 82 with a wide rim 84. In use, the rim 84 engages the flexible connector 52 and secures it in place. A physician may tighten the set screw 78 using a tightening tool (not shown) configured to engage the hex head 82. In the embodiment shown, the set screw 78 is a snap-off set screw. Accordingly, when a proper amount of torque is reached, the hex head 82 may snap off the set-screw 78, thereby notifying the physician that the set screw 78 is sufficiently tight. Although the device 50 is described using a snap-off set screw 78, other attachments methods could be used. For example, in some embodiments, the set screw 78 does not include a snap-off hex head, but may be tightened to a desired torque using a torque wrench. In other embodiments, instead of a set screw, the flexible connector 52 is held in place by a nut attachable to the screws 74. In one example, the nut is a lock-nut. In other embodiments, a lock-washer or clamping connector is used. Still other devices also could be used to secure the flexible connector 52 to the screw 74, as would be apparent to one skilled in the art. In other embodiments, the vertebral fasteners 54, 56 may include cables, crimps, loops, press fits, tethers, and adhesives, among others.

Implanting the vertebral stabilizing system 50 may be accomplished using, for example, a posterior, posterior-lateral, or lateral approach. First, a small incision may be created in the patient's skin for access to the pedicle region. The pedicle region of the vertebrae 14, 16 may be visualized directly or may be visualized with radiographic assistance. Using a drill, a suitably sized hole may be formed into the pedicle of one of the vertebrae 14, 16 in the area between the transverse process 22 and the superior articular process 30. The screw 74 may be driven partially into the hole, while leaving a portion extending outwardly for connection to the flexible connector 52. The drilling process may be repeated for the other of the vertebrae 14, 16 at a proper distance from the first hole, and a second screw 74 may be driven into the hole.

The flexible connector 52 may then be placed over the two screws 74 so that the two screws protrude through the apertures 66 at the ends 62, 64 of the flexible connector 52. In some embodiments, if it is desired to apply the flexible connector 52 either in tension or in compression, and thereby apply loading to the vertebrae, the flexible connector 52 may be either compressed or stretched while being placed over the two screws 74. Once the flexible connector 52 is in place, set screws 78 may be threaded onto the screws 74. The set screws 78 are threaded onto the screw 74 until they engage the flexible 52 connector with a desired torque. While threading, the rim 84 engages and presses against the flexible connector 52.

The flexible connector 52 may be placed directly adjacent the vertebrae 14, 16, or alternatively, may be spaced from the vertebrae 14, 16. In some embodiments, placement of the flexible connector 52 directly adjacent the vertebrae 14, 16 may impart specific characteristics to the flexible connector 52. In some examples, the flexible connector 52 may be spaced from the vertebrae 14, 16. Accordingly even when the vertebral column is in flexion, causing the spine to bend forward, the first and second vertebral fasteners 54, 56 maintain a line of sight position, so that the flexible connector 52 extends only along a single axis, without bending. In other examples, after placement, the flexible connector 52 may contact portions of the vertebrae 14, 16 during the flexion process. For example, during flexion, the vertebrae 14, 16 may move so that the first and second vertebral fasteners 54, 56 do not have a line of sight position. Accordingly, the flexible connector 52 may be forced to bend around a protruding portion of the vertebrae. This may impart additional characteristics to the flexible connector 52. For example, because the flexible connector 52 would effectively contact the spinal column at three locations (its two ends 62, 64 and somewhere between the two ends), its resistance to extension might be increased.

In the exemplary embodiments described, the flexible connector 52 is the only component extending from one vertebral fastener 54, 56 to the other. This may be referred to as a single flexible connector. This single flexible connector may be contrasted with conventional systems that employ more than one connector extending between attachment points, such as systems with one component connected at the attachment points and another component extending between attachment points. Because it employs a single flexible connector 52, the vertebral stabilizing system 50 disclosed herein may be easier and quicker to install, may be less complex, and may be more reliable than prior devices.

It should be noted however, that a spinal column may employ the flexible connector 50 to extend across a first vertebral space, with a second flexible connector extending across a second vertebral space. Accordingly, more than one vertebral stabilizing system 50 may be used in a spinal column. In some instances where more than one stabilizing system is use, the first and second vertebral spaces may be adjacent. In alternative embodiments, a vertebral stabilizing system 50 may have a single flexible connector with a length allowing it to extend across more than one intervertebral space, with or without connecting to an intermediate vertebra.

In certain anatomies, the vertebral stabilizing system 50 may be used alone to provide decompression or compression to a single targeted facet joint or to relieve pressure on a particular side of the intervertebral disc, such as a herniation area. However, in some instances, a second vertebral stabilizing system may be installed on the opposite lateral side of the vertebrae 14, 16, across from the vertebral stabilizing system 50. Use of first and second vertebral stabilizing systems may provide more balanced support and equalized stabilization. The second vertebral stabilizing system may be substantially similar to system 50 and therefore will not be described in detail.

The vertebral stabilizing system 50, as installed, may flexibly restrict over-compression of the vertebrae 14, 16, thereby relieving pressure on the intervertebral disc 12 and the facet joint 44. In addition, the vertebral stabilizing system 50 may flexibly restrict axial over-extension of the intervertebral disc 12 and the facet joint 44. By controlling both compression and extension, the vertebral stabilizing system 50 may reduce wear and further degeneration. The flexible connector 52 may also dampen the forces on the intervertebral disc 12 and facet joint 44 during motion such as flexion and extension. Because the flexible connector 52 may be positioned relatively close to the natural axis of flexion, the vertebral stabilizing system 50 may be less likely to induce kyphosis as compared to systems that rely upon inter-spinous process devices to provide compressive and tensile support. Additionally, the system 50 may be installed minimally invasively with less dissection than the inter-spinous process devices of the prior art. Furthermore, an inter-pedicular system can be used on each lateral side of the vertebrae 14, 16, and may provide greater and more balanced stabilization than single inter-spinous process devices.

FIGS. 5-7 show alternative embodiments of the flexible connector 52. For example, FIG. 5 shows a flexible connector 52′ having the apertures 66 and the optional reinforcing members 60 as described above. However, in this embodiment, instead of an hourglass shape, the flexible connector includes straight sides, with an array of flexibility-affecting holes 86 disposed between the two apertures 66. In the embodiment shown in FIG. 5, the flexibility-affecting holes 86 are a series of rectangular-shaped through-holes aligned in a row. In other embodiments, the flexibility-affecting holes are not through holes, but instead are cavities extending only part way through the flexible connector. The flexibility-affecting holes are sized and spaced to provide a desired level of resistance to extension, compression, and torsion. By adjusting the height, width, and depth of the holes, the flexible connector 52′ may provide any desired level of flexibility. For example, if more flexibility is desired in under axial loads, the width of the rectangular holes may be increased. Further, if more flexibility is desired in torsion, the height of the rectangular holes may be increased. The edges and corners of the flexibility-affection holes 86 may be rounded to reduce stress-risers and distribute stress through the flexible connector 52′. This may prolong the life of the flexible connector 52′, allowing it to be effective for lengthy periods of time.

FIG. 6 shows another exemplary embodiment of the flexible connector. In this embodiment, a flexible connector 52″ includes a central hole 88 disposed in the center of the flexible connector 52″. As described above, the flexible connector 52″ may be designed to provide desired levels of resistance to extension, deflection, and torsion. In the embodiment of FIG. 6, the flexible connector 52″ does not provide high resistance to torsion and compression. Accordingly, This embodiment allows a high level of torsional and compressive displacement. In this embodiment, the flexible connector 52″ includes walls 90 on each side of the central hole 88 that are bowed outwardly from the end portions. Accordingly, under a compressive load, the walls 90 would further bow outwardly, imparting low levels of support to the vertebrae in the compressive direction. However, the flexible connector 52″ may provide a greater degree of resistance to extension thereby providing support to the vertebrae by limiting the chance of overextension, and thereby protection the vertebrae.

FIG. 7 shows an additional embodiment of the flexible connector. In this embodiment, the flexible connector 52′″ is a solid connector having straight sides without additional holes. Even still, the width, thickness, and the material used may provide a desired resistance to compression, extension, or torsion. In this embodiment, the flexible connector 52′″ may include high resistance to compression, extension, and torsion. It should be noted that the flexible connector embodiments shown are exemplary only, as the flexible connectors may designed to provide any desired resistance to loading.

It should be noted that in some embodiments, the flexible connector 52 may be configured so that orientation in one direction provides one set of stabilizing properties to the vertebrae, while orienting the flexible connector 52 in the other direction would provide a second set of stabilizing properties. In such an embodiment, the body 58 of the flexible member may be asymmetrically shaped.

Although disclosed as being used at the posterior areas of the spine, the flexible connector may also be used in the anterior region of the spine to support the anterior column. In such a use, the flexible connector may be oriented adjacent to and connect to the anterior column, and may span a vertebral disc space.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,”“top,” “upper,” “lower,” “bottom,” “left,” “right,” “cephalad,” “caudal,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

We claim:

1. A system for flexibly stabilizing a vertebral column in tensile and compressive loading by connecting a first and a second vertebrae, the system comprising:
first means for connecting to the first vertebra;
second means for connecting to the second vertebra; and
a flexible connector configured to extend from and connect the first means to the second means, the flexible connector including first and second apertures for respectively attaching to the first and second means for connecting, each aperture including a reinforcement member therein.

2. The system of claim 1, wherein the flexible connector includes flexibility affecting holes formed therein.

3. The system of claim 1, wherein the reinforcement member is a grommet.

4. The system of claim 1, wherein the flexible connector includes a first end and a second end that are rounded to reduce occurrence of distress to tissue about the first and second means.

5. The system of claim 1, wherein the flexible connector forms an hourglass shape.

6. The system of claim 1, wherein first and second means include first and second pedicle screws.

7. The system of claim 1, wherein first and second means include first and second set screws.

8. The system of claim 7, wherein the first and second set screws include a rim configured to engage the flexible connector.

9. The system of claim 1, wherein only the flexible connector extends from the first means to the second means.

10. The system of claim 1, wherein the flexible connector is disposed to come into contact with one of the first and second vertebrae during flexion of the first vertebra relative to the second vertebra.

11. A system for flexibly stabilizing a vertebral column in tensile and compressive loading by connecting a first and a second vertebrae, the system comprising:
first means for connecting to the first vertebra;
second means for connecting to the second vertebra; and
a flexible connector configured to extend from and connect the first means to the second means, the flexible connector including flexibility affecting holes formed therein.

12. The system of claim 11, wherein the flexible connector includes a first aperture and a second aperture for connection to the first and second means for connecting.

13. The system of claim 12, including a reinforcement member associated with at least one of the first and second apertures.

14. The system of claim 13, wherein the reinforcement member is a grommet.

15. The system of claim 11, wherein the flexible connector includes a first end and a second end that are rounded to reduce occurrence of distress to tissue about the first and second means.

16. The system of claim 11, wherein the flexibility affecting holes are cavities.

17. The system of claim 11, wherein the first and second means include first and second pedicle screws

18. The system of claim 11, wherein the first and second means include first and second set screws.

19. A system for flexibly stabilizing a vertebral column in tensile and compressive loading by connecting a first and a second vertebra, each of the first and second vertebra having a transverse process and a superior articular process, the system comprising:
first means for connecting to the first vertebra, the first means being configured to be secured to the first vertebra in a position between the transverse process and the superior articular process;
second means for connecting to the second vertebra, the second means being configured to be secured to the second vertebra in a position between the transverse process and the superior articular process; and
a flexible connector having a single material and being configured to span a distance between the first means and the second means, the flexible connector being configured to provide a stabilizing tensile force and a stabilizing compressive force to the first and second vertebrae.

20. The system of claim 19, including flexibility affecting holes formed therein.

21. The system of claim 19, wherein the flexible connector includes a first aperture and a second aperture for connection to the first and second means for connecting.

22. The system of claim 21, including a reinforcement member associated with at least one of the first and second apertures.

23. The system of claim 19, wherein the flexible connector includes a first end and a second end that are rounded to reduce occurrence of distress to tissue about the first and second means.

24. The system of claim 19, wherein the flexible connector forms an hourglass shape.

25. The system of claim 19, wherein the first and second means include first and second pedicle screws.

26. The system of claim 19, wherein the first and second means include first and second set screws.

27. A method of flexibly stabilizing vertebrae on a spinal column, comprising:
accessing a pair of pedicles on the vertebrae;
installing vertebral fasteners on the pair of pedicles;
placing a flexible connector to extend around an exterior of the vertebral fasteners to connect the pair of pedicles; and
securing the flexible connector to the vertebral fasteners,
wherein installing the vertebral fasteners is accomplished with the transverse processes and the superior articular processes of the vertebrae intact; and wherein only the flexible connector extends the distance between and attached to the pair of pedicles.

28. The method of claim 27, wherein installing vertebral fasteners includes drilling a hole and securing a screw within the hole.

29. The method of claim 27, wherein the flexible connector include apertures, and wherein placing a flexible connector includes arranging the flexible connector so that the vertebral fasteners extend through the apertures.

30. The method of claim 27, wherein securing the flexible connector to the vertebral fastener includes tightening a set screw.

31. A system for flexibly stabilizing a vertebral column in tensile and compressive loading by connecting a first and a second vertebrae, the system comprising:
first means for connecting to the first vertebra;
second means for connecting to the second vertebra; and
a single flexible connector configured to extend from and connect the first means to the second means, the flexible connector including a first end and a second end that is rounded to reduce occurrence of distress to tissue about the first and second means.

32. The system of claim 31, wherein the flexible connector includes a first aperture and a second aperture for connection to the first and second means for connecting.

33. The system of claim 32, including a reinforcement member associated with at least one of the first and second apertures.

34. The system of claim 31, wherein the flexible connector is reinforced.
